(19) United States
(12) Patent Application Publication
(10) Pub. No.: US 2020/0129429 A1
(43) Pub. Date: Apr. 30, 2020

(54) BIOCOMPATIBLE AND BIODEGRADABLE EMULSIONS AND COMPOSITIONS, AND METHODS OF USE THEREOF

(71) Applicant: VIVAVAX INC., Toronto (CA)

(72) Inventors: Qi YANG, Etobicoke (CA); Winston DANG, Windsor (CA); Chad Gorman GROVES, Oshawa (CA); Monir Riasad FADLE AZIZ, Toronto (CA); Ruby Rose RAFANAN, Toronto (CA)

(73) Assignee: Vivavax Inc., Toronto, ON (CA)

(21) Appl. No.: 16/493,063

(22) PCT Filed: Mar. 15, 2018
(86) PCT No.: PCT/IB2018/051750

§ 371 Date: Sep. 11, 2019

Related U.S. Application Data
(60) Provisional application No. 62/471,596, filed on Mar. 15, 2017.

(51) Int. Cl. A61K 9/113; A61K 39/00; A61K 39/39; A61K 47/12; A61K 47/14; A61K 47/42

(52) U.S. Cl. CPC A61K 9/113; A61K 39/0015; A61K 39/39; A61K 2039/55566; A61K 47/14; A61K 47/42; A61K 47/12

(57) ABSTRACT

Emulsions and layered compositions for improving thermal resistance of a substrate, such as a therapeutic or prophylactic medicinal substrate, are provided. Also provided are methods and kits for making the emulsions and layered compositions of the invention. The layered compositions and emulsions can be used to extend the shelf life of a product.

### CROSS REFERENCE TO RELATED APPLICATION

This application claims priority under 35 U.S.C. § 119 to U.S. Provisional Patent Application No. 62/471,596 filed on Mar. 15, 2017, and the disclosure of which is herein incorporated by reference in its entirety.

### FIELD OF THE INVENTION

The invention relates to emulsions including biodegradable or biocompatible molecules that have heat-absorbing properties. The emulsions of the invention are useful for, e.g., maintaining a stable temperature range of heat-sensitive substrates, such as pharmaceuticals and specialized foods, during transportation and protecting such substrates from thermal fluctuations in the surrounding environment.

### BACKGROUND OF THE INVENTION

The Food and Drug Administration of U.S. (FDA) specifies that a number of perishable foods and medical products have narrow temperature ranges within which they need to be stored at. Especially in the case of heat-sensitive drugs, such as biologics and specialty medications, exposure to temperatures outside of their mandated range (temperatures either too low or too high) can lead to a loss of efficacy.

Efforts to monitor and track the temperature of these heat-sensitive goods during transportation and storage have been implemented by pharmaceutical and food distributors. However, these solutions serve as remedies rather than preventative measures. Especially in the case of pharmaceutical products, temperatures during transport and storage are critical in the preservation of their quality and functionality. Consequences of heat excursions can range between medical non-compliance to serious harm to the unknowing patient. Indeed, the World Health Organization (WHO) identified vaccine handling during transportation and storage as a point for major improvement in 2016 (See, e.g., World Health Organization, “Vaccines Stockouts and Use of Vaccines in a Controlled-Temperature Chain” Monitoring Results: Goals, strategic objectives, and indicators, pages 115-128 available at http://www.who.int/immunization/global_vaccine_action_plan/gvap_2016_secretariat_report_stockouts.pdf.)

Current systems of temperature maintenance depend largely on mechanical methods, which are high cost, low reliability, and often dependent on electrical power supply. Thus, pharmaceutical manufacturers have begun developing medications that are inherently more heat-stable. For small molecule therapeutics and chemical therapeutics, this method has shown improved heat-stability outcomes for a number of products. However, because of the highly sensitive nature of newer biological therapeutics to its surroundings (temperature, ions, pH, etc.), improvements in the inherent heat-resistance of these therapeutics are largely restricted to enhancements in the heat-tolerability of the suspension or solution of these drug products.

### SUMMARY OF THE INVENTION

The invention solves this problem by providing a composition that can be mixed with, or used to suspend, a medical product or its ingredients for improved heat-stability and extended shelf life of the product. More specifically, the invention solves this problem by providing biocompatible and biodegradable emulsions and layered compositions containing endothermic molecules that are amphiphilic, and may contain chemical or biological substrates. The inventive emulsions and layered compositions protect such substrates contained within and/or surrounded by the emulsion or layered composition from fluctuations in temperature, eliminating or reducing the need for temperature maintenance that depends largely on mechanical methods. Emulsions and layered compositions of the invention are particularly useful with heat-sensitive substrates, such as pharmaceutical products and perishable foods, and can enhance their heat-resistance. By improving the heat-resistance of such substrates, the shelf-life and stability during storage can be improved.

In one aspect, the invention relates to an emulsion comprising:
(i) an inner disperse aqueous phase comprising albumin, at least one benzoic acid derivative, and at least one salt;
(ii) a lipophilic phase comprising at least one polyunsaturated fatty acid, a nonionic surfactant, and a polyglycerol ester of a fatty acid; and
(iii) optionally, an outermost continuous aqueous phase comprising an aqueous solvent, a nonionic surfactant, and at least one salt
   wherein the lipophilic phase is an intermediate disperse lipophilic phase when the optional continuous aqueous phase is present, and wherein the lipophilic phase is an outer continuous lipophilic phase when the optional continuous aqueous phase is absent.

In another aspect, the invention relates to a kit comprising:
(i) a first aqueous composition comprising albumin, at least one benzoic acid derivative, and at least one salt;
(ii) a lipophilic composition comprising at least one polyunsaturated fatty acid, a nonionic surfactant and a polyglycerol ester of a fatty acid; and
(iii) a second aqueous composition comprising an aqueous solvent, a nonionic surfactant, and at least one salt.

In yet another general aspect, the invention relates to method of improving thermal resistance of a substrate or thermally insulating a substrate by encapsulating the substrate in an emulsion, the method comprising:
(i) mixing the substrate with a first aqueous composition to form an aqueous mixture, the first aqueous composition comprising albumin, at least one benzoic acid derivative, and at least one salt;
(ii) mixing the aqueous mixture obtained in step (i) with a lipophilic composition to obtain a water-in-oil emulsion, the lipophilic composition comprising at least one polyunsaturated fatty acid, a nonionic surfactant and a polyglycerol ester of a fatty acid; and
(iii) optionally mixing the water-in-oil emulsion obtained in step (ii) with a second aqueous composition to obtain the water-oil-water emulsion, the second aqueous composition comprising an aqueous solvent, a nonionic surfactant, and at least one salt.

And in yet another general aspect, the invention relates to a layered composition comprising:
(i) a lipophilic layer comprising at least one polyunsaturated fatty acid, a nonionic surfactant and a polyglycerol ester of a fatty acid;
(ii) a first aqueous layer comprising an aqueous solvent, a nonionic surfactant, and at least one salt; and
(iii) optionally, a second aqueous layer comprising albumin, at least one benzoic acid derivative, and at least one salt, wherein when present, the second aqueous layer is separated from the first aqueous layer by the lipophilic layer.

In yet another general aspect, the invention relates to a method of making an emulsion, the method comprising:
(i) mixing a first aqueous composition with a lipophilic composition to obtain a water-in-oil emulsion, wherein the first aqueous composition comprises albumin, at least one benzoic acid derivative, and at least one salt, and the lipophilic composition comprises at least one polyunsaturated fatty acid, a nonionic surfactant and a polyglycerol ester of a fatty acid; and
(ii) optionally mixing the water-in-oil emulsion obtained in step (i) with a second aqueous composition to obtain a water-oil-water emulsion, the second aqueous composition comprising an aqueous solvent, a nonionic surfactant, and at least one salt.

The invention also relates to methods of thermally insulating a substrate by encapsulating the substrate in an emulsion or layered composition of the invention, or surrounding the substrate with an emulsion or layered composition of the invention.

Other aspects, features and advantages of the invention will be apparent from the following disclosure, including the detailed description of the invention and its preferred embodiments, and the appended claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown in the drawings and described in the following detailed description of the invention.

In the drawings:

FIG. 1 shows the chemical structure of polyglycolides, wherein m is an integer of 2 to 12, which can be used in an emulsion of the invention to achieve slower heat transfer between the external environment and an emulsion of the invention, or substrate encapsulated therein;

FIGS. 2A-2C show the chemical structures of benzoic acid derivatives that can be used in an emulsion of the invention; FIG. 2A shows the structure of a benzoic acid molecule substituted with a hydrocarbon chain having a free terminal hydroxyl group, wherein n is an integer of 1 to 12; FIG. 2B shows the structure of a benzoic acid molecule substituted with a polyether chain having a free terminal hydroxyl group, wherein x is an integer of 1 to 12; FIG. 2C shows the structure of a dibenzoic acid derivative, wherein y is an integer of 1 to 12;

FIG. 3 show the structure of a sterol-substituted saccharide that can be used in an emulsion of the invention, specifically a cholesterol-bearing pullulan (CHP);

FIGS. 4A-4G show schematic illustrations of emulsions according to embodiments of the invention; oil and/or water droplets of the distinct (inner) phases can be of any spherical or oval shape; the emulsions of the invention can be single emulsions or double emulsions; FIG. 4A shows an illustration of a W/O/W double emulsion, wherein each oil/lipophilic droplet contains a single water/aqueous droplet; FIG. 4B shows an illustration of a W/O/W double emulsion, wherein each oil/lipophilic droplet contains multiple water/aqueous droplets; FIG. 4C shows a W/O/W double emulsion, wherein each droplet contains multiple water/aqueous droplets of variable size; FIG. 4D shows an illustration of W/O single emulsion, wherein the continuous oil phase contains multiple water/aqueous droplets of variable size; FIG. 4E shows an illustration of an W/O single emulsion, wherein the continuous oil phase contains multiple water/aqueous droplets of substantially the same size; FIG. 4F shows an illustration of a O/W single emulsion, wherein the continuous water phase contains multiple oil/lipophilic droplets of variable size; and FIG. 4G shows an illustration of a O/W single emulsion, wherein the continuous water phase contains multiple oil/lipophilic droplets of variable size;

FIG. 5 shows the results of the study described in Example 1;

FIG. 6 shows the results of study described in Example 2; and

FIG. 7 shows the results of the study described in Example 3.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which have been included in the present specification is for the purpose of providing context for the invention. Such discussion is not an admission that any or all of these matters form part of the prior art with respect to any inventions disclosed or claimed.

Unless otherwise defined, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural references unless the context clearly dictates otherwise.

As used herein, the term “substrate” refers to any naturally occurring or synthetic molecule or compound. A substrate can also be a container or package. Preferably, a substrate is a chemical or biological compound. Examples of substrates include, but are not limited to, pharmaceutical products, food products, and other biological products, such as nucleic acids; proteins; antibodies; live, dead, or attenuated virus; active, inactive, or partially inactive microorganisms or parts thereof; an active or inactive metabolic product of an organism; a blood sample; a living or dead organism, etc. In particular embodiments of the invention, a substrate is a medicinal substrate, such as a therapeutic or prophylactic/preventative substrate. As used herein, the term “therapeutic substrate” refers to a substrate that has medicinal uses in treating a disease, disorder, or condition in humans or animals. Therapeutic substrates include pharmaceutical products and certain biological products. As used herein, the terms “prophylactic substrate” and “preventative substrate” refer to a substrate that has medicinal uses in preventing a disease, disorder, or condition in humans or animals. Prophylactic substrates include, for example, vaccines. In those embodiments in which a substrate is a container or package, the container or package can further include a medicinal substrate, e.g., therapeutic or prophylactic substrate.

Preferably, a substrate as used herein is heat-sensitive and is stable in a temperature range of 0° C. to 25° C., more preferably in a temperature range of 2° C. to 8° C. According to embodiments of the invention, encapsulating or surrounding a substrate in an emulsion of the invention improves heat stability of the substrate, such that the substrate can tolerate temperatures outside of the range in which the substrate is typically stable, such as temperatures outside a range of 2° C. to 8° C. (i.e., above 8° C. and/or below 2° C.), and even outside a range of 0° C. to 25° C. (i.e., above 25° C. and/or below 0° C.). In a particularly preferred embodiment, a substrate is a heat-sensitive therapeutic or prophylactic substrate.

The invention relates to an emulsion that improves the thermal resistance of an encapsulated substrate. Without wishing to be bound by any theories, it is believed that the components of the emulsion absorb thermal energy through endothermic configurational or conformational changes, thus insulating a substrate encapsulated by the emulsion and protecting the substrate itself from absorbing the thermal energy.

In one aspect, the invention relates to an emulsion comprising:
(i) an inner disperse aqueous phase comprising albumin, at least one benzoic acid derivative, and at least one salt;
(ii) a lipophilic phase comprising at least one polyunsaturated fatty acid, a nonionic surfactant and an emulsifier; and
(iii) optionally, an outermost continuous aqueous phase comprising an aqueous solvent, a nonionic surfactant, and at least one salt
   wherein the lipophilic phase is an intermediate disperse lipophilic phase when the optional continuous aqueous phase is present, and wherein the lipophilic phase is an outer continuous lipophilic phase when the optional continuous aqueous phase is absent.

An “emulsion” as used herein has the typical meaning known in the art, and refers to a composition or formulation that is a mixture of two or more liquids that are normally immiscible. In an emulsion, one or more liquid phases (the inner or disperse phases) are dispersed throughout another liquid phase (the continuous or outer phase). The boundary between each phase is known as an “interface.” Emulsions are typically named from the innermost phase to the outermost phase. For example, oil (or lipophilic compositions) and water (or aqueous based compositions) can form an emulsion, including an oil-in-water (O/W) emulsion, wherein the oil or lipophilic composition is the dispersed or inner phase and the water or aqueous composition is the dispersion medium, i.e., the continuous or outer phase. Oil (or lipophilic compositions) and water (or aqueous-based compositions) can also form a water-in-oil (W/O) emulsion, wherein water or aqueous composition is the dispersed or inner phase and the oil or lipophilic composition is the dispersion medium, i.e., the continuous or outer phase. Such emulsions containing two layers or phases are referred to as “single emulsions.” An emulsion can also be a “double emulsion” containing additional layers or phases, such as a water-in-oil-in-water (W/O/W) emulsion, or an oil-in-water-in-oil (O/W/O) emulsion. In a W/O/W emulsion, an immiscible oil or lipophilic phase exists between two separate water phases, wherein the two water or aqueous phases can be the same or different. In an O/W/O emulsion, an immiscible water phase separates two different oil phases.

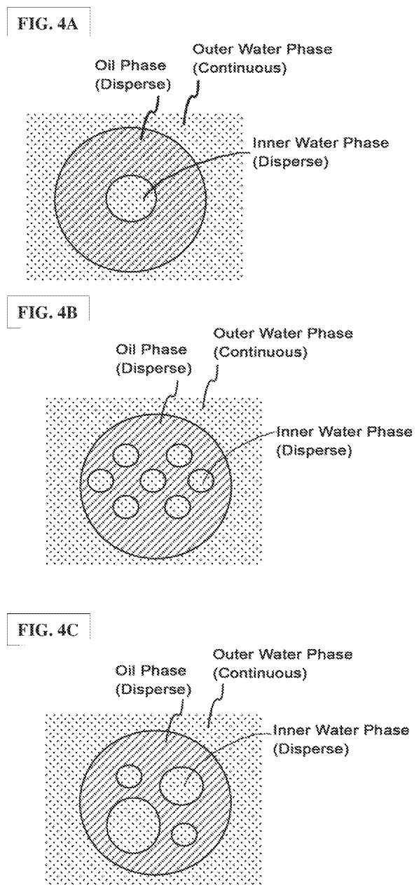

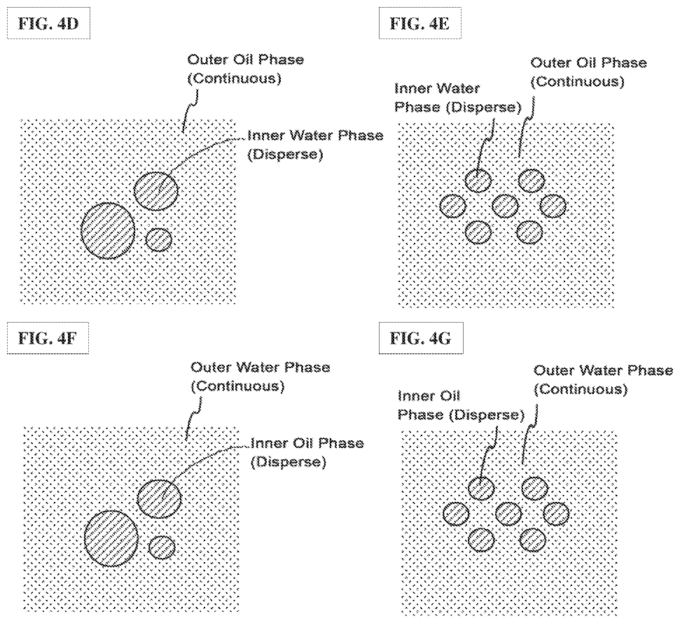

According to embodiments of the invention, an emulsion can be a single emulsion, or a double emulsion. In some embodiments, an emulsion of the invention is a single emulsion, preferably water-in-oil (W/O) single emulsion comprising an inner disperse aqueous phase and an outer continuous lipophilic phase. See, e.g., FIGS. 4D-4E. In some embodiments, an emulsion of the invention is a double emulsion, preferably a W/O/W double emulsion comprising an inner disperse aqueous phase, an intermediate disperse lipophilic phase, and an outer continuous aqueous phase. Preferably, an emulsion of the invention is a double emulsion, and more preferably a W/O/W double emulsion. See FIGS. 4A-4C for schematic illustrations of W/O/W double emulsions according to embodiments of the invention.

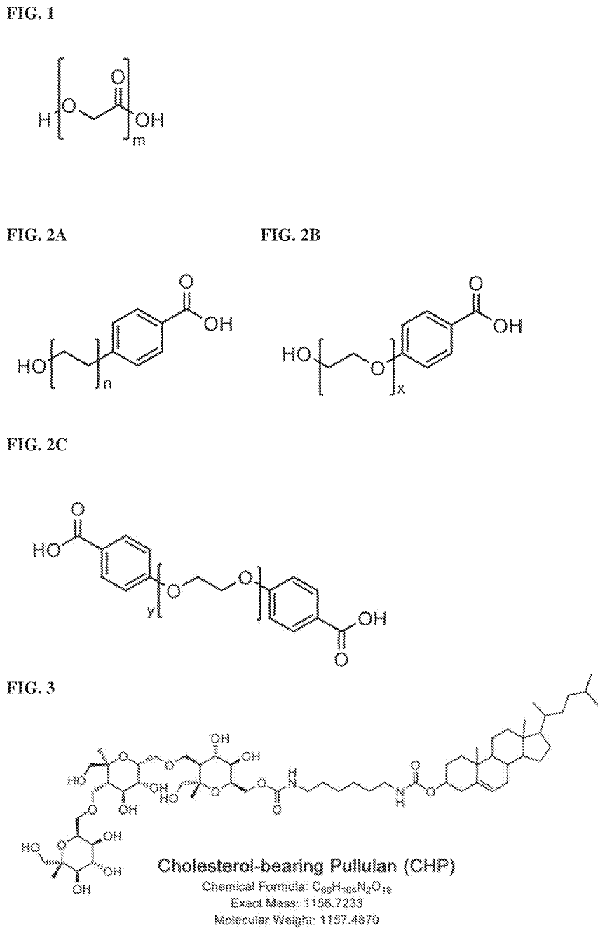

According to embodiments of the invention, an emulsion comprises components that are biodegradable and/or biocompatible, and which are able to absorb heat slowly from the surroundings and/or release heat slowly to the surroundings, thus providing insulating effects in an environment where there is a temperature gradient or temperature fluctuation. At least one of three types of molecules is included in the emulsions of the invention to achieve this insulating effect, namely polyglycolides (see, e.g., FIG. 1), benzoic acid derivatives (see, e.g., FIG. 2A-2C), and sterol-substituted saccharides (see, e.g., FIG. 3). An emulsion of the invention can be used to surround or encapsulate one or more substrates to reduce exposure of the substrate to sudden heat influxes (or temperature increase) from the environment or surroundings. An emulsion of the invention can also be used to surround or encapsulate one or more substrates to reduce exposure of the substrate to sudden decreases in temperature in the environment or surroundings.

According to embodiments of the invention, an emulsion comprises at least one benzoic acid derivative in at least one of the aqueous and oil phases, preferably in an aqueous phase, more preferably in at least an inner or disperse aqueous phase. A benzoic acid derivative is included to increase thermal stability. As used herein, “benzoic acid derivative” refers to a compound comprising at least one benzoic acid molecule substituted with at least one hydrocarbon chain or polyether chain. A benzoic acid derivative also includes a dibenzoic acid derivative, in which two benzoic acid molecules are linked via a polyether chain or hydrocarbon chain. A benzoic acid derivative can optionally be further substituted with one or more suitable substituent groups including, but not limited to single atoms, halogens, straight-chain or branched alkyl, alkenyl, alkynyl, aryl, alkoxy, aryloxy, aromatic, hydroxy, carboxy, nitro, cyano, isocyano, thiocyano, isothiocyano, or azide groups, wherein the substituent groups optionally can aid in polymerization of the monomer units. Preferably, a benzoic acid derivative is capable of polymerization with other benzoic acid derivatives of the same or different structure. Preferably, a benzoic acid derivative is 1,4-substituted, meaning that the carboxyl group is at position 1 of the ring and the additional substitution (i.e., polyether chain, hydrocarbon chain, etc.) is at position 4 of the ring. Benzoic acid derivatives can be added to an emulsion of the invention as monomer units or as polymer units, in which one or more of the same or different benzoic acid derivative monomer units are polymerized together to form longer polymer chains. Benzoic acid derivatives can also be added to an emulsion of the invention as monomer units, and polymerize into longer polymer chains within the emulsion.

In a preferred embodiment, a benzoic acid derivative is 1,4-substituted with a hydrocarbon chain having a terminal alcohol group, or a polyether chain having a terminal alcohol group. In another preferred embodiment, a benzoic acid derivative is a dibenzoic acid derivative in which two benzoic acid molecules are linked via a polyether or hydrocarbon chain. Any of these dibenzoic acid and benzoic acid derivatives can optionally be further substituted with one or more suitable substituent groups.

In particular embodiments of the invention, a benzoic acid derivative is a compound of formula (I), a compound of formula (II), or a compound of formula (III):

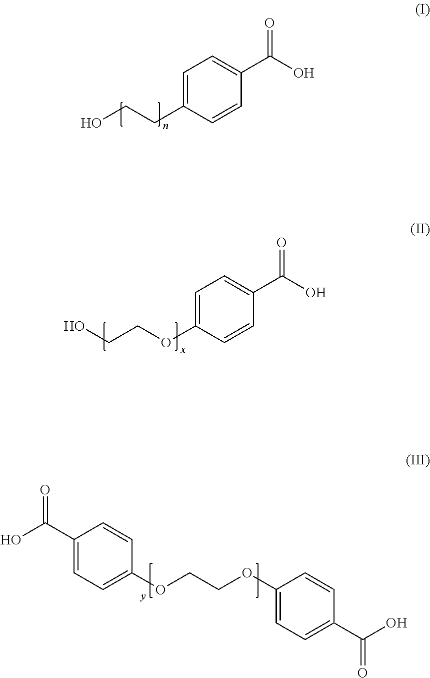

wherein each of x, y, and n is independently an integer of 1 to 12. See FIGS. 2A, 2B and 2C. Specific examples of dibenzoic acid derivatives suitable for use in the invention include, but are not limited to, 1,8-bis(p-carboxyphenoxy)-3,6-dioxaoctane and 4,4′-((3, 6, 9, 12-tetraoxatetradecane-1,14-diyl)bis(oxy))dibenzoic acid.

In a particularly preferred embodiment of the invention, the benzoic acid derivative is 1,8-bis(p-carboxyphenoxy)-3,6-dioxaoctane.

In preferred embodiments of the invention, a benzoic acid derivative is included in an inner disperse aqueous phase of an emulsion.

In some embodiments of the invention, a benzoic acid derivative, such as 1,8-bis(p-carboxyphenoxy)-3,6-dioxaoctane, is optionally included in an outer continuous aqueous phase of an emulsion.

According to embodiments of the invention, an emulsion contains an albumin in at least one of the aqueous and oil phases, preferably in an aqueous phase, more preferably in an inner disperse aqueous phase. Any albumin from any human or mammalian source known in the art in view of the present disclosure can be used, including, but not limited to, bovine serum albumin (BSA) and human serum albumin. The albumin can be obtained from any known commercial source, or recombinantly produced. Albumin acts as a protein emulsifier, which can provide structural stability to an emulsion or composition.

An emulsion of the invention includes at least one salt in at least one of the aqueous and oil phases, preferably in at least one aqueous phase, more preferably in an inner disperse aqueous phase and an outermost continuous aqueous phase. Any salt can be used in an emulsion of the invention including, but not limited to sodium salts, magnesium salts, etc., such as sodium chloride, etc. The salt functions to provide ionic stabilization to the emulsion. Preferably, the salt is sodium chloride. When a salt is included in more than one phase, the identity of the salt can be the same or different. For example, when a salt is included in both the inner disperse aqueous phase and the outer continuous phase, the identity of the salt can be the same, e.g., sodium chloride, in both phases, or the identity of the salt can be different, e.g., sodium chloride in the inner disperse aqueous phase and potassium chloride in the outer continuous phase.

An emulsion of the invention can also include a polyglycolide in one or more of the aqueous or lipophilic phases. Preferably, a polyglycolide is included in at least one of the lipophilic phase and the outer continuous aqueous phase. Most preferably, a polyglycolide is included in at least the outer continuous aqueous phase. Examples of polyglycolides suitable for use in the invention include those shown in FIG. 1, wherein m is an integer of 2 to 12, i.e., 2, 3, 4, 5, 6, 7, 8, 9, 10, 11, or 12.

In a particular embodiment, an outer continuous phase of an emulsion of the invention further comprises a polyglycolide of formula (IV):

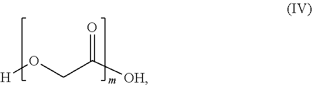

wherein m is an integer of 2 to 12.

Other components that can be included in an emulsion of the invention in at least one of the aqueous and oil/lipophilic phases to enhance or increase the thermal insulating effects of the emulsion are monosaccharides or polysaccharides with sterol-group substitutions. A monosaccharide or polysaccharide can be substituted with one or more sterol groups. When substituted with multiple sterol groups, the sterol groups can be the same or different. Examples of saccharides that can be used include, but are not limited to glucose, glycogen, and pullulan. Examples of sterol groups that can be used include, but are not limited to cholesterol. An example of a preferred sterol-substituted polysaccharide for use in the invention is cholesterol-bearing pullulan (see FIG. 3).

Emulsions of the invention can further include at least one fatty acid, preferably in the lipophilic phase, as a solvent. Preferably the fatty acid is a monounsaturated or polyunsaturated fatty acid, more preferably a C₁₆-C₂₂ polyunsaturated fatty acid, such as a C₁₆, C₁₇, C₁₈, C₁₉, C₂₀, C₂₁, or C₂₂ monounsaturated or polyunsaturated fatty acid. Examples of fatty acids suitable for use in the invention include, but are not limited to linoleic acid and oleic acid. In one embodiment, the fatty acid included in the lipophilic phase is linoleic acid. In another embodiment, the fatty acid included in the lipophilic phase is oleic acid. In yet another embodiment, both linoleic acid and oleic acid are included in the lipophilic phase.

Other components suitable for use in an emulsion of the invention include surfactants, emulsifiers, and additional solvents which are commonly known to and used by those skilled in the art. Surfactants and emulsifiers can be present in one or more of the aqueous and oil phases in addition to any of the other components described herein.

Examples of emulsifiers and surfactants suitable for use in the invention include non-ionic surfactants, such as sorbitan monooleate (e.g., Span80®), polysorbate 80 (polyoxyethylene glycol sorbitan monooleate, e.g., Tween80®), polysorbate 60 (polyoxyethylene glycol sorbitan monostearate, e.g., Tween60®), polysorbate 40 (polyoxyethylene sorbitan monopalmitate, e.g., Tween40®), polysorbate 20 (polyoxyethylene sorbitan monolaurate, e.g., Tween20®), and other polysorbates; and polyglycerol esters of fatty acids, such as polyglycerol riconoleate.

Examples of solvents suitable for use in an aqueous phase of an emulsion of the invention include, but are not limited to, water and aqueous alcohol solvents, such as aqueous ethanol.

Preferably, each of the components of an emulsion of the invention is at least one of biodegradable and biocompatible. As used herein, “biodegradable” means that a component can be degraded by enzymes or co-activators in metabolizing processes that occur naturally in the body of a human or an animal, into metabolites or other breakdown products that are non-toxic or not harmful to the human or animal. As used herein, “biocompatible” means that a component is either harmless or non-toxic to the body of a human or an animal, or is present in an amount that is below the tolerable amount published by the U.S. Food and Drug Administration and/or the European Medicines Agency. For example, each of the albumin, benzoic acid derivative, polyunsaturated fatty acid, nonionic surfactant, polyglycerol ester of a fatty acid, aqueous solvent, and salt is independently biodegradable and/or biocompatible.

One of ordinary skill in the art will be able to select the appropriate components for each phase of an emulsion in order to achieve the desired insulating and heat-resistant effects in view of the present disclosure. As general guidance, about 80% of more of the components of an emulsion of the invention preferably have a denaturing temperature above 60° C., a melting temperature above 25° C., or a boiling temperature above 60° C.

In some embodiments, an emulsion of the invention comprises 0.5% to 10% (w/v) of a benzoic acid derivative in the inner disperse aqueous phase, such as 0.5%, 1%, 2%, 3%, 4%, 5%, 6%, 7%, 8%, 9%, or 10% (w/v) of a benzoic acid derivative. In a preferred embodiment, an emulsion of the invention comprises 1% to 3% (w/v) of a benzoic acid derivative in the inner disperse aqueous phase. In a particular embodiment, an emulsion of the invention comprises 0.5% to 10% (w/v), more preferably 1% to 3% (w/v) of monomeric 1,8-bis(p-carboxyphenoxy)-3,6-dioxaoctane in the inner disperse aqueous phase.

In some embodiments, an emulsion of the invention comprises 0.02% to 5% (w/v), such as 0.02%, 0.025%, 0.05%, 0.1%, 0.2%, 0.5%, 1%, 1.2%, 1.4%, 1.6%, 1.8%, 2%, 3%, 4%, or 5% (w/v) of albumin in the inner aqueous disperse phase, preferably 0.025% (w/v) of albumin.

In some embodiments, an emulsion of the invention comprises 0.5% to 5% (w/v) of at least one salt, such as 0.5%, 1%, 2%, 3%, 4%, or 5% (w/v) of at least one salt in the inner disperse aqueous phase, preferably 2% (w/v). In a particular embodiment, an emulsion of the invention comprises 2% (w/v) of sodium chloride in the inner aqueous disperse phase.

In some embodiments, an emulsion of the invention comprises 0% to 10% (w/v), such as 0%, 1%, 2%, 3%, 4%, 5%, 6%, 7%, 8%, 9% or 10% (w/v), preferably 2% (w/v) of ethanol in the inner disperse aqueous phase.

In some embodiments, an emulsion of the invention comprises 0% to 10% (w/v), such as 0%, 1%, 2%, 3%, 4%, 5%, 6%, 7%, 8%, 9% or 10% (w/v), preferably 1% to 2% (w/v) of a polysorbate, such as polysorbate 80, in the inner disperse aqueous phase.

In some embodiments, an emulsion of the invention comprises 0% to 5% (w/v), such as 0%, 0.1%, 0.2%, 0.3%, 0.4%, 0.5%, 0.6%, 0.8%, 0.9%, 1%, 2%, 3%, 4%, or 5% (w/v), preferably 0.01% (w/v) of sodium hydroxide in the inner disperse aqueous phase.

In some embodiments, an emulsion of the invention comprises 10% to 95% (w/v), such as 10%, 20%, 30%, 40%, 50%, 60%, 70%, 80%, 90%, or 95% (w/v), preferably 15% to 80% (w/v) of at least one polyunsaturated fatty acid in the lipophilic phase. In a particular embodiment, an emulsion of the invention comprises 10% to 95% (w/v), more preferably 15% to 80% (w/v) of linoleic acid in the lipophilic phase.

In some embodiments, an emulsion of the invention comprises 0.5% to 10% (w/v), such as 0.5%, 1%, 2%, 3%, 4%, or 5%, preferably 0.5% to 5% (w/v) of a non-ionic surfactant in the lipophilic phase. In a particular embodiment, an emulsion of the invention comprises 0.5% to 10% (w/v), such as 0.5%, 1%, 2%, 3%, 4%, or 5%, preferably 0.5% to 5% (w/v) of sorbitan monooleate in the lipophilic phase.

In some embodiments, an emulsion of the invention comprises 0.1% to 10% (w/v), such as 0.1%, 0.5%, 1%, 2%, 3%, 4%, 5%, 6%, 7%, 8%, 9%, or 10% (w/v) of a polyglycerol ester of a fatty acid in the lipophilic phase. In a particular embodiment, an emulsion of the invention comprises 0.1% to 10% (w/v), such as 0.1%, 0.5%, 1%, 2%, 3%, 4%, 5%, 6%, 7%, 8%, 9%, or 10% (w/v) of a polyglycerol polyricinoleate in the lipophilic phase.

In some embodiments, an emulsion of the invention comprises 0% to 5% (w/v), such as 0%, 1%, 2%, 3%, 4%, or 5% (w/v) of a sterol-substituted saccharide in the lipophilic phase. In a particular embodiment, an emulsion of the invention comprises 0% to 5% (w/v), such as 0%, 1%, 2%, 3%, 4%, or 5% (w/v) of a cholesterol bearing pullulan in the lipophilic phase.

In some embodiments, an emulsion of the invention comprises 0% to 80% (w/v), such as 0%, 10%, 20%, 30%, 40%, 50%, 60%, 70% or 80% (w/v), preferably 20% to 70% (w/v) of oleic acid in the lipophilic phase.

In some embodiments, an emulsion of the invention comprises 1% to 10% (w/v), such as 1%, 2%, 3%, 4%, 5%, 6%, 7%, 8%, 9%, or 10% (w/v), preferably 2% to 5% (w/v) of a nonionic surfactant in the outer continuous aqueous phase. In a particular embodiment, an emulsion of the invention comprises 1% to 10% (w/v), such as 1%, 2%, 3%, 4%, 5%, 6%, 7%, 8%, 9%, or 10% (w/v), preferably 2% to 5% (w/v) of polysorbate 80 in the outer continuous aqueous phase.

In some embodiments an emulsion of the invention comprise 1% to 10% (w/v), such as 1%, 2%, 3%, 4%, 5%, 6%, 7%, 8%, 9%, or 10% (w/v), preferably 4% to 8% (w/v) of ethanol in the outer continuous aqueous phase.

In some embodiments, an emulsion of the invention comprises 2% to 10% (w/v), such as 2%, 3%, 4%, 5%, 6%, 7%, 8%, 9%, or 10% (w/v), preferably 2% to 5% (w/v) of at least one salt in the outer continuous aqueous phase. In a particular embodiment, an emulsion of the invention comprises 2% to 10% (w/v), such as 2%, 3%, 4%, 5%, 6%, 7%, 8%, 9%, or 10% (w/v), preferably 2% to 5% (w/v) of sodium chloride in the outer continuous aqueous phase.

In some embodiments, an emulsion of the invention comprises 0% to 10% (w/v), such as 0%, 1%, 2%, 3%, 4%, 5%, 6%, 7%, 8%, 9%, or 10% (w/v), preferably 2% to 5% (w/v) of a sterol-substituted polysaccharide in the outer continuous aqueous phase. In a particular embodiment, an emulsion of the invention comprises 0% to 10% (w/v), such as 0%, 1%, 2%, 3%, 4%, 5%, 6%, 7%, 8%, 9%, or 10% (w/v), preferably 2% to 5% (w/v) of cholesterol bearing pullulan in the outer continuous aqueous phase.

In some embodiments, an emulsion of the invention comprises 0% to 5% (w/v), such as 0%, 1%, 2%, 3%, 4%, or 5% (w/v), preferably 1% to 2% (w/v) of a benzoic acid derivative in the outer continuous aqueous phase. In a particular embodiment, an emulsion of the invention comprises 0% to 5% (w/v), such as 0%, 1%, 2%, 3%, 4%, or 5 (w/v), preferably 1% to 2% (w/v) of monomeric 1,8-bis(p-carboxyphenoxy)-3,6-dioxaoctane in the outer continuous aqueous phase.

In some embodiments, an emulsion of the invention has a pH of 5 to 8.6, such as pH 5, 5.5, 6.0, 6.5, 7.0, 7.5, 8.0, or 8.5.

The ratio by volume of each phase in an emulsion of the invention is not particularly limited. In particular embodiments of a W/O/W double emulsion of the invention, a ratio by volume of the inner disperse aqueous phase to intermediate disperse lipophilic phase to outer continuous aqueous phase is 1:1:1 to 1:2:5, such as 1:1:1, 1:2:2, 1:2:3, 1:2:3, or 1:2:5, preferably 1:2:2 to 1:2:5. For example, a W/O/W double emulsion in which the ratio of phases is 1:2:2 by volume has an inner disperse aqueous phase that is 1 mL, an intermediate disperse lipophilic phase that is 2 mL, and an outer continuous aqueous phase that is 2 mL.

The diameter of droplets (aqueous and lipophilic) in an emulsion of the invention is not particularly limited. In particular embodiments of an emulsion of the invention, a diameter of the droplets constituting the inner disperse aqueous phase is at least 15 nm to 2 mm, such as 15 nm, 30 nm, 50 nm, 75 nm, 100 nm, 200 nm, 400 nm, 600 nm, 800 nm, 1 mm, 1.2 mm, 1.4 mm, 1.6 mm, 1.8 mm, or 2 mm. In other particular embodiments of an emulsion of the invention, a diameter of the droplets constituting the intermediate disperse lipophilic phase is at least 15 nm to 1 cm, such as 15 nm, 50 nm, 100 nm, 200 nm, 400 nm, 600 nm, 800 nm, 1 mm, 2 mm, 3 mm, 4 mm, 5 mm, 6 mm, 7 mm, 8 mm, 9 mm, or 1 cm. When a lipophilic droplet contains multiple aqueous droplets, each of the aqueous droplets can have substantially the same diameter, or each aqueous droplet can have a different diameter. One of ordinary skill in the art will be able to determine/measure the diameter in view of the present disclosure. Each phase can be visibly distinct or non-distinct from other phases or layers, and immiscible with other layers or phases.

In some embodiments, an emulsion of the invention further comprises a substrate in at least one phase of the emulsion. The terms “encapsulate,” “surround,” “immerse,” or “disperse” when used with respect to a substrate refer to a substrate being distributed in one or more phases of an emulsion. In a typical embodiment, a substrate will be dispersed or encapsulated in one phase of an emulsion. However, embodiments in which a substrate is distributed over multiple phases in a uniform or non-uniform manner are also contemplated by the invention. It is also possible for multiple substrates to be encapsulated or dispersed in one or more phases of an emulsion of the invention. In such embodiments in which multiple different substrates are encapsulated in an emulsion of the invention, each substrate can be present in the same or different phase from the other different substrates. A substrate can be surrounded, immersed, encapsulated, or dispersed in a uniform or non-uniform manner, covering the substrate entirely or partially. A substrate can be inserted or embedded into one or multiple layers or phases of the emulsion, to achieve maximal protection from heat or other features of the local environment for the desired use or application. In embodiments when a substrate is, e.g., a container, an emulsion of the invention can surround the exterior of the container or the interior of the container. A substrate that is e.g., a therapeutic or prophylactic substrate can also be placed in a container or other suitable packaging, and the exterior of the container or other suitable packaging can be surrounded with an emulsion of the invention.

In some embodiments of the invention, an emulsion can further comprise one or more additional aqueous and/or lipophilic phases. One of ordinary skill in the art will be able to determine the appropriate number of phases in order to achieve the desired effect in view of the present disclosure.

In a particular embodiment of the invention, an emulsion comprises:
(i) an inner disperse aqueous phase comprising albumin, 1,8-bis(p-carboxyphenoxy)-3,6-dioxaoctane, and sodium chloride;
(ii) an intermediate disperse lipophilic phase comprising linoleic acid, sorbitan monooleate, and polyglycerol polyricinoleate; and
(iii) an outer continuous aqueous phase comprising aqueous ethanol, polysorbate 80, and sodium chloride.

Any of the components in one or more phases of an emulsion of the invention can be present in monomeric, oligomeric, or polymeric form, or a combination thereof. The components of an emulsion, whether in monomeric, oligomeric, or polymeric form, can form associations with one or more of the same or different components via ionic, covalent, or hydrogen-bonding interactions, and the associations can be reversible or irreversible. Components of the emulsion can also form ionic, covalent, or hydrogen bonding interactions with the substrate encapsulated therein. Typically, such interactions with a substrate are reversible, and preferably at least about 80% to 98% of these interactions are reversible. Any such interactions that occur with a substrate preferably do not affect the properties of the substrate, such as activity or efficacy, by more than about 30% relative to the unencapsulated substrate, e.g., the substrate in buffer or aqueous solution.

It is believed that the thermal insulation properties of an emulsion of the invention result from the formation of oligomers and/or polymers, as such forms are more stable over a range of temperatures, including temperatures outside of the tolerable temperature range of heat-sensitive substrates encapsulated therein. Thus, it is believed the components capable of existing in an oligomeric or polymeric form, such as benzoic acid derivatives, typically exist in the emulsion in oligomeric or polymeric form.

In some embodiments of the invention, components of an emulsion can have certain advantageous properties, namely biologically-relevant properties, such as enzymatic activities, adjuvanting activities, immunosuppressant activities, coagulating or anticoagulating activities, being or degrading into a naturally-occurring metabolite, or being a proponent involved in the rhythmic signal propagations of the body, in amounts and ways that should not alter the typical functioning of the body (e.g., body parts or organs) when ingested or injected into the bloodstream in the body of a human or an animal. In particular embodiments, about 60% (w/v) of an emulsion of the invention possesses such biologically relevant properties. Examples of components which have such biologically relevant properties that can be included in the emulsions of the invention include sterol-substituted polysaccharides, albumin, and polyglycolides. Another desirable function of molecules with enzymatic activities is that they are capable of stabilizing other components or emulsifying the mixtures.

The specifications described here are general guidelines for replicating the inventive emulsion. One of ordinary skill in the art will recognize that the particular components, ratio of components, and arrangement of phases based on the type of emulsion can be varied for the desired application. The components of each phase should be selected, and the phases should be arranged such that the emulsion acts as a buffer to any substrate encapsulated or dispersed therein and first intakes thermal energy upon heat exposure, is able to store thermal energy for long periods of time without significant changes to the physical or chemical characteristics of the emulsion, is able to delay the transfer of such thermal energy to any encapsulated substrates, or is able to release thermal energy slowly when heat transfer to the external environment is favorable. Examples of physical and chemical properties that should be considered for these purposes include hydrophobicity, solubility, molecular arrangement, crystallinity, rate of degradation, melting point temperature, boiling point temperature, degradation profile and rate, and elasticity.

Preferably, about 70%-80% (w/v) of an emulsion of the invention is comprised of biodegradable components, meaning that such components can be degraded by enzymes or co-activators in metabolizing processes that occur naturally in the body of a human or an animal, into metabolites or other breakdown products that are non-toxic or not harmful to the human or animal. The remaining about 20%-30% (w/v) of an emulsion of the invention comprises biocompatible components, meaning that such components, in whole or in part, are either harmless or non-toxic to the body of a human or an animal, or are present in amounts well below the tolerable amounts published by the U.S. Food and Drug Administration and/or the European Medicines Agency.

In some embodiments, about 10-40% (w/v) of the emulsion comprises molecules or components capable of polymerizing with identical or different molecules or components through functional end-groups or side-groups, such as benzoic acid derivatives. Optionally, modifications to side chain groups and functional units can be made, such as the alteration, addition, or reduction of single atoms, halogens, straight-chain or branched alkyl, alkenyl, alkynyl, aryl, alkoxy, aryloxy, aromatic, hydroxy, carboxy, nitro, cyano, isocyano, thiocyano, isothiocyano, or azide groups to one or more repeating units. In general, the benzoic acid derivatives used in the invention should have monomer chain lengths of between 1 to 30 carbons. When used in an emulsion of the invention, the majority of these polymerizing molecules may polymerize to be homogenously or heterogeneously oligomeric or polymeric molecular structures or networks, which is believed to provide heat insulating properties, and thus increase heat-resistance.

The biodegradation behavior of the components in an emulsion of the invention is ideally controlled by bulk erosion, which can be triggered by certain changes in the local environment. By means of listing examples, and with no intention of limiting the scope of the invention, such changes in the local environment include changes in available thermal or kinetic energy, changes in local or global pH, changes in local or global humidity, changes in exposure to other components of the emulsion, and changes in exposure to an encapsulated substrate. Certain components, particularly oligomeric or polymeric molecules, can be highly susceptible to fragmentation by hydrolysis, with monomeric units most ideally connected using ester, peptide, anhydride, and phosphoester bonds that hydrolyze into biocompatible breakdown products over several different steps. For example, when an emulsion of the invention is ingested by or injected into the bloodstream of a human or animal, there should be no detectable levels of any molecules included in the formulation about 24 hours post-consumption.

Emulsions can be formed by any method known in the art in view of the present disclosure. Emulsion formation typically requires some energy input, such as by stirring or shaking to mechanically mix the otherwise immiscible phases. Components can be added and/or mixed to form an emulsion in any form, e.g., solid liquid, etc. One of ordinary skill in the art will be familiar with techniques and methods for preparing emulsions. For example, when a substrate is encapsulated in an emulsion of the invention, additional hardware may be needed for processing and/or providing shape to the final product. The term “final product” is used here to mean an emulsion of the invention further comprising a substrate encapsulated or dispersed therein. By way of listing examples and not limiting the proposed applications of the invention, hardware for processing can be a mixing chamber, a sheer or pressure homogenizer, a channel-based microfluidics device, or devices for membrane emulsification, sonication, etc. or a series of different machineries or devices or combinations thereof. The hardware for providing shape to the final product can be an open or closed container for holding the substrate surrounded with the formulation therein, such as a sleeve or mold, or other devices that perform a similar or substantially the same function as a container or mold. The hardware for providing shape to the final product can also include an absorbent material to which the substrate surrounded by the formulation can be bound, such as a tissue, cloth, or membrane network.

In a preferred embodiment, an emulsion of the invention is producing using a microfluidics device for mixing.

In another general aspect, the invention relates to a layered composition comprising:
(i) a lipophilic layer comprising at least one polyunsaturated fatty acid, a nonionic surfactant and a polyglycerol ester of a fatty acid;
(ii) a first aqueous layer comprising an aqueous solvent, a nonionic surfactant, and at least one salt; and
(iii) optionally, a second aqueous layer comprising albumin, a benzoic acid derivative, and at least one salt, wherein when present, the second aqueous layer is separated from the first aqueous layer by the lipophilic layer.

According to embodiments of the invention, a layered composition comprises at least two immiscible layers: an aqueous layer and a lipophilic layer. A layered composition of the invention optionally further comprises a third layer, preferably an aqueous layer. When a layered composition of the invention includes a third, preferably aqueous layer, the aqueous layers are separated by the lipophilic layer, such that the composition has the structure aqueous layer-lipophilic layer-aqueous layer. Any of the components and amounts described herein with respect to the emulsions of the invention can be used in the layered compositions of the invention.

Each phase of a layered composition can be visibly distinct or non-distinct from other phases or layers, and immiscible with other layers or phases. Each layer can have a thickness of about 10 nm to 1 cm, such as 10 nm, 1 mm, 5 mm, 10 mm, or 1 cm. The thickness can be uniform or non-uniform throughout each layer, or in spherical form surrounding an encapsulated substrate. For example, the thickness of each layer can be about 10 nm to 1 mm; or 1 mm to 1 cm. The order of layers in the arrangement can be varied or reversed depending on the specific application case of the formulation.

In some embodiments, a layered composition of the invention further comprises a substrate dispersed in one or more layers (e.g., aqueous and/or lipophilic layers) of the composition. In other embodiments, a layered composition of the invention is used to surround a substrate, e.g., a container, wherein the container optionally further comprises an additional substrate, such as a medicinal substrate (e.g., therapeutic or prophylactic substrate).

Without wishing to be bound by any theories, it is believed that the thermally protective benefits of the emulsions and layered compositions of the result from the tendency of long linear polymers and co-polymers to interact and form molecular networks that are both physically and thermally stable. For example, the networking effects of polymers are believed to allow for absorption and trapping of heat from the local environment, while their closely packed structures and local saturation of solution are believed to minimize large structural changes that may affect the reversibility of the heat absorbency or binding interactions with an encapsulated substrate.

In another general aspect, the invention relates to a kit comprising:
(i) a first aqueous composition comprising albumin, a benzoic acid derivative, and at least one salt;
(ii) a lipophilic composition comprising at least one polyunsaturated fatty acid, a nonionic surfactant and a polyglycerol ester of a fatty acid; and
(iii) a second aqueous composition comprising an aqueous solvent, a nonionic surfactant, and at least one salt.

Compositions of a kit of the invention can be combined to form an emulsion of the invention, with or without a substrate, using any of the methods described herein. Alternatively, compositions of the invention can be used to form a layered composition of the invention, with our without a substrate, using any of the methods described herein. A kit can further include a substrate in a separate composition. Alternatively, at least one of the first aqueous composition, lipophilic composition, and second aqueous composition can further comprise a substrate. A kit can include also include additional compositions (aqueous or lipophilic) for forming additional layers or phases in an emulsion or layered composition of the invention. A kit can also include hardware for processing the compositions to form an emulsion of the invention, such as a mixing chamber, a sheer or pressure homogenizer, a channel-based microfluidics device, or devices for membrane emulsification, sonication, etc., or a series of different machineries or devices or combinations thereof.

In a particular embodiment of the invention, a kit further comprises a microfluidics device, such as a channel based microfluidics device.

In yet another general aspect, the invention relates to a method of making an emulsion of the invention. According to embodiments of the invention, the method comprises:
(i) mixing a first aqueous composition with a lipophilic composition to obtain a water-in-oil emulsion, wherein the first aqueous composition comprises albumin, at least one benzoic acid derivative, and at least one salt, and the lipophilic composition comprises at least one polyunsaturated fatty acid, a nonionic surfactant and a polyglycerol ester of a fatty acid; and
(ii) optionally mixing the water-in-oil emulsion obtained in step (i) with a second aqueous composition to obtain a water-oil-water emulsion, the second aqueous composition comprising an aqueous solvent, a nonionic surfactant, and at least one salt.

The aqueous and lipophilic compositions can be mixed together using any method known in the art in view of the present disclosure for preparing emulsions. Preferably, the mixing is performed using a microfluidics device, such as a channel based microfluidics device.

In some embodiments, a substrate is mixed with one or more of the aqueous and lipophilic compositions, preferably the first aqueous composition, prior to forming the emulsion. For example, the first aqueous composition can be mixed with a substrate prior to mixing the first aqueous composition with the lipophilic composition in order to encapsulate the substrate in an inner aqueous phase of the obtained emulsion.

In yet another general aspect, the invention relates to a method of improving thermal resistance of a substrate by encapsulating the substrate in an emulsion. According to embodiments of the invention, a method comprises:
(i) mixing the substrate with a first aqueous composition to form an aqueous mixture, the first aqueous composition comprising albumin, at least one benzoic acid derivative, and at least one salt;
(ii) mixing the aqueous mixture obtained in step (i) with a lipophilic composition to obtain a water-in-oil emulsion, the lipophilic composition comprising at least one polyunsaturated fatty acid, a nonionic surfactant and a polyglycerol ester of a fatty acid; and
(iii) optionally mixing the water-in-oil emulsion obtained in step (ii) with a second aqueous composition to obtain a water-oil-water emulsion, the second aqueous composition comprising an aqueous solvent, a nonionic surfactant, and at least one salt.
   Any method known in the art in view of the present disclosure can be used for mixing to obtain the emulsion. Preferably, the mixing is performed using a microfluidics device, such as a channel based microfluidics device.

As used herein, the term “thermal resistance” as used with respect to a substrate, means the ability of a substrate to delay an increase in its internal temperature in the presence of external heat, or to delay a decrease in its internal temperature in the presence of a temperature decrease in the external environment or surroundings. In one embodiment, thermal resistance results in a reduced sensitivity of a substrate to temperature changes and fluctuations, such that the substrate has improved stability outside of a temperature range in which the substrate is typically stable. In another embodiment, thermal resistance results in decreased heat exposure of a substrate. In another embodiment, thermal resistance results in temperature stabilization of a substrate, such as during storage or transport.

The terms “insulating” and “insulate” as used herein with respect to a substrate refer to protecting the substrate by reducing heat transfer from the substrate to the environment (e.g., when the environmental temperature is too low), or from the environment to the substrate (e.g., when the environmental temperature is too high). Thus, the insulating effect is preferably thermal insulation.

Encapsulating or dispersing a substrate in an emulsion or layered composition of the invention has many advantages and can be used for many purposes, as discussed in greater detail below.

In one embodiment, encapsulating a substrate or surrounding a substrate with an emulsion or layered composition of the invention can be used to insulate the substrate.

For example, some preferred applications of the invention include, but are not limited to, encapsulating an active medical ingredient (i.e., therapeutic substrate or prophylactic substrate) with or without excipients, mixing with an ingestible or injectable drug or protein product, emulsifying with medical compounds to provide a fundamental diluent or suspension upon which drug-specific improvements can be made, mixing with human or animal samples that are aqueous or dry, surrounding a medical goods package with the emulsion, or layered composition and enclosing a viable food item with a sealable sleeve containing the formulation.

A potential useful application for the disclosed emulsion or compositions related to surrounding a substrate, is a design where components of the emulsion or layered composition are separated according to hydrophobicity. An example of a substrate to be surrounded could be a package of pharmaceutical pills that are sensitive to fluctuations in local temperature. In a particular embodiment with respect to use of a layered composition of the invention, the package containing the pills can be embedded into a further packaging entity containing two distinct layers, the inner layer of which includes an aqueous phase solution and the outer layer of which includes a lipophilic or oil phase solution. Optionally, a further aqueous outer layer can be included. For example, the outer lipophilic or oil phase layer can act as an insulation layer against incoming heat from the local environment. This layer can contain a number of oligomeric and polymeric molecules that are able to flex and adopt higher-energy conformations upon exposure to incoming heat, and retain that thermal energy until a later time at which the local environment is lower in thermal energy, before transferring the energy back to the environment and letting its component molecules revert back into lower-energy conformations. The inner aqueous phase would contain components of the formulation that are water-soluble, and are emulsifiers that thicken the solution to obstruct the flow of thermal energy from the lipophilic layer through it, into the pill packaging. By delaying the flow of heat into the core pill packaging, the outer lipophilic layer will be forced to retain its thermal energy and/or release its heat energy into the local environment instead of transferring the heat energy into the aqueous layer and further into the pills.

Another preferred application of the invention is to surround a heat-sensitive substrate, such as a delicate vegetable, in a re-sealable sleeve enclosed and filled with the emulsion or layered composition of the invention. When exposed to heat during transport, the emulsion or composition can absorb and retain the thermal energy, which can then be slowly released later during storage in a colder environment such as a cold room or refrigerator where the vegetable would be more susceptible to over-freezing.

Another preferred application of the invention is to mix a therapeutic or prophylactic substrate or composition containing a therapeutic or prophylactic substrate with an emulsion or layered composition, preferably an emulsion of the invention. The therapeutic or prophylactic substrate or composition can be designed to naturally segregate into an existing layer or phase of the emulsion or layered composition, or its own independent phase. Depending on the emulsion or composition design, it can either be stripped away from the therapeutic or prophylactic substrate or composition before ingestion or administration to a human or animal, or alternatively it can be ingested or administered along with the therapeutic or prophylactic substrate or composition, particularly since preferably about 90-100% of the components are either biocompatible or biodegradable.

For example, a therapeutic or prophylactic substrate, along with a diluent or buffer, if desired, can be incorporated into the innermost aqueous phase of an emulsion, which is then made into small droplets. The aqueous droplets are then single or multiply dispersed into a second phase of the formulation, which can be a lipophilic or oil-based phase. This emulsion can then be further encapsulated into the outermost aqueous layer, which is believed to provide the greatest insulating effect out of the three layers. For an ingestible or injectable therapeutic or prophylactic, the emulsion does not have to be removed before drug administration, and can be administered along with the therapeutic or prophylactic substrate.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the following Examples, test results and appended claims.

### EXAMPLES

With the sole intention of illustrating certain principles and practices of the invention, and by no way limiting the scope of the invention, we provide here examples of specific embodiments of the invention.

### Example 1: Preparation of an Emulsion of the Invention Encapsulating an Enzyme Substrate

The enzyme substrate alkaline phosphatase (AP), which performs cleavage activities optimally at physiological temperature, was conjugated to a goat anti-murine IgG antibody. The conjugated AP enzyme was encapsulated in an emulsion according to an embodiment of the invention.

The formulation included three phases. The first, innermost phase, was a water-based phase including 5% ethanol, 1% polysorbate 80, and 1% monomeric 1,8-bis(p-carboxyphenoxy)-3,6-dioxaoctane. The second, middle phase, was an oil-based phase including 78% linoleic acid, 5% sorbitan monooleate, and 0.5% polyglycerol polyricinoleate.

The third, outermost phase, was another water-based phase including 5% polysorbate 80, 5% ethanol, 5% cholesterol-bearing pullulan sugar, and 4% sodium chloride. The three phases were added in the order described to uniformly surround AP, in a ratio of 1:2:5 by volume (first, second, third phases, respectively). More specifically, the first water-based phase was mixed with the oil-based phase to form an W/O emulsion. Then, the W/O emulsion was mixed with the outermost water phase to provide the final emulsion. The method used for surrounding the conjugated AP enzyme with consecutive layers was vigorous mixing. Alternatively, a channel-based microfluidics device can be used instead of vigorous mixing. The conjugated AP enzyme encapsulated by the emulsion was heated to 37° C. As a control, the conjugated AP enzyme in phosphate buffered saline (PBS) was heated to 37° C. The activity of the conjugated AP enzyme was measured at 0 hour, 1 hours, and 3 hours post-heating. More specifically, the enzymatic activity of the conjugated AP enzyme was measured as follows:
(1) Samples containing the conjugated AP enzyme were collected at three time points (0, 1, 3 hours) post-heating at 37° C.
(2) A direct ELISA method was used for enzyme activity characterization.
   a. The assay plate was coated with a monoclonal murine IgG antibody (“coating antibody”)
   b. The goat anti-murine IgG AP-conjugated antibody (“sample”) was added to the plate and incubated to allow crosslinking with the coating antibody
   c. The plate was washed and the substrate to AP (which is pNPP, or p-nitrophenyl phosphate) was added and incubated (“substrate”)
(3) AP-cleaved pNPP gives off a yellow color. A spectrophotometer was used to read the plate at an absorption of 405 nm (yellow detection), and background noise was checked to be minimal at 560 nm.

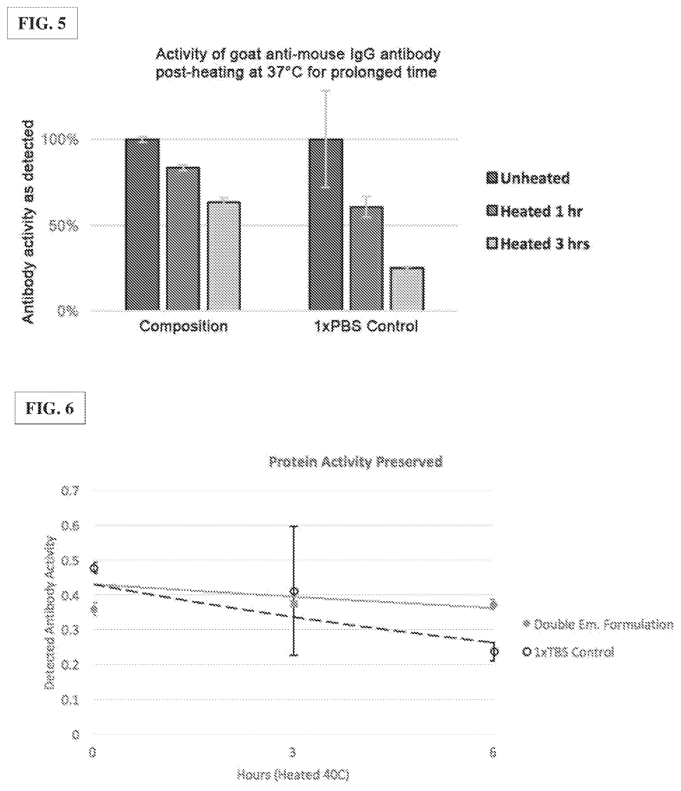

The results are shown in FIG. 5. The results demonstrate that this embodiment of the invention was able to maintain the conjugated AP enzyme activity by approximately 130% more effectively after 1 hour of heating at 37° C., and 220% after 3 hours of heating, compared to the control condition (where the conjugated AP enzyme was mixed into 1×PBS solution at the same proportions). It was also demonstrated that the activity of the conjugated AP enzyme had less variability when mixed with this embodiment of the invention, compared to the control condition.

Accordingly, the results demonstrate that encapsulating an enzyme with an emulsion of the invention can reduce the heat susceptibility of the enzyme and preserve the enzymatic activity of the enzyme, even after heating at 37° C., as compared to the observed enzymatic activity after heating in PBS (FIG. 5).

### Example 2: Preparation of an Emulsion of the Invention Encapsulating an Antibody

A mouse IgG antibody was encapsulated in a double emulsion according to an embodiment of the invention. The double emulsion included three phases: an innermost aqueous phase, a middle oil-based phase, and an outer aqueous phase.

Specifically, the mouse IgG antibody was homogenized with 0.025% (w/v) albumin, 1% (w/v) monomeric 1,8-bis(p-carboxyphenoxy)-3,6-dioxaoctane, 2% (w/v) sodium chloride, 0.01% (w/v) sodium hydroxide, and 5% (v/v) ethanol in an aqueous phase. The aqueous phase was then emulsified through vigorous mixing with an outer hydrophobic (oil-based) phase containing 5% (w/v) sorbitan monooleate, 0.5% (w/v) polyglycerol polyricinoleate, 2% (w/v) cholesterol-bearing pullulan, 70% (w/v) oleic acid, and 15% linoleic acid. The resulting single emulsion was then emulsified with an outermost aqueous phase, which contains 1% (w/v) monomeric 1,8-bis(p-carboxyphenoxy)-3,6-dioxaoctane, 4% (w/v) sodium chloride, and 5% (w/v) polyethylene glycol sorbitan monooleate. The ratio by volume of the three phases from the first (inner) phase, second phase, to third (outer) phase, respectively, was 1:2:2.

The resulting emulsion was heated to 40° C. for 6 hours, and the activity of the mouse IgG antibody was measured. More specifically, the functional preservation of the mouse IgG antibody was measured as follows:
(1) Samples containing the mouse IgG antibody was collected at three time points (0, 3, 6 hours) post-heating at 37° C.
(2) An indirect ELISA method was used for the detection of functional preservation.
   a. The assay plate was coated with a polyclonal goat anti-murine IgG (H+L) antibody (“coating antibody”)
   b. The mouse IgG antibody (“sample”) was added to the plate and incubated to allow crosslinking with the coating antibody
   c. A gamma-chain specific goat anti-mouse IgG AP-conjugated antibody (“detection antibody”) was added to the plate to allow crosslinking with the sample
   d. The plate was washed and the substrate to AP (which is pNPP, or p-nitrophenyl phosphate) was added and incubated
(3) AP-cleaved pNPP was detected as described in Example 1.

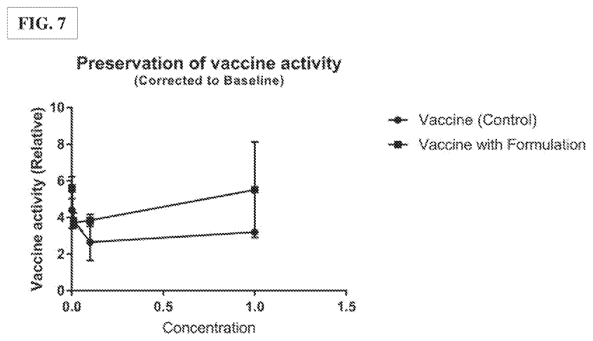

The results are shown in FIG. 6. It was demonstrated that this embodiment of the invention was able to maintain the functional activity of the antibody by approximately 160% more effectively after 6 hours of heating at 37° C., compared to the control condition (where the conjugated AP enzyme was mixed into 1×TBS solution at the same proportions). Accordingly, the results demonstrate that encapsulation of the mouse IgG antibody in an emulsion of the invention provided for improved function and recognition of the antibody under heating at 40° C. for over 6 hours, as compared to the function measured after heating the antibody in Tris-buffered saline (TBS).

### Example 3: Preparation of an Emulsion of the Invention Encapsulating a Vaccine

A commercially available MMRII (measles, mumps, rubella) vaccine was encapsulated by a double emulsion according to an embodiment of the invention. Specifically, the MMRII vaccine was homogenized with 0.025% (w/v) albumin, 1% (w/v) monomeric 1,8-bis(p-carboxyphenoxy)-3,6-dioxaoctane, 1.9% (w/v) sodium chloride, 0.5% (w/v) sodium hydroxide, and 1% (v/v) polyethylene glycol sorbitan monooleate in an aqueous phase. The obtained aqueous composition was then emulsified through vigorous mixing with an outer hydrophobic phase containing 70% (w/v) oleic acid and 15% linoleic acid. The resulting single emulsion was then emulsified with an outermost aqueous phase containing 4% (w/v) sodium chloride, 5% ethanol, and 5% (w/v) polyethylene glycol sorbitan monooleate. The ratio between the three phases was 1:1:1 from innermost to outermost.

The obtained emulsion was heated to 37° C. for one hour. For the “vaccine control,” the vaccine diluted with 1×PBS (phosphate buffered saline) instead of with the emulsion. The functional preservation of the MMRII vaccine was then measured as follows:
(1) Samples of the MMRII vaccine (diluted by volume at 1/1, 1/5, and 1/10) were collected at 1 hour post-heating at 37° C.
(2) A sandwich ELISA method was used for the detection of functional preservation.
   a. The assay plate was coated with an anti-measles mouse IgG antibody (“coating antibody”)
   b. Diluted samples of the MMRII vaccine (“sample”) were added to the plate and incubated to allow crosslinking with the coating antibody
   c. Another anti-measles mouse IgG antibody (“primary antibody”) was added to the plate and incubated to allow crosslinking with the sample
   d. A gamma-chain specific goat anti-mouse IgG AP-conjugated antibody (“secondary antibody”) was added to the plate to allow crosslinking with the primary antibody
   e. The plate was washed and the substrate to AP (which is pNPP, or p-nitrophenyl phosphate) was added and incubated
(3) AP-cleaved pNPP was detected as described in Example 1.

The results are shown in FIG. 7. It was demonstrated that this embodiment of the invention had a specific effect that maintained the functional activity of the MMRII vaccine under heating conditions, compared to the control condition (where the vaccine was mixed into 1×PBS solution at the same proportions). Accordingly, the results demonstrate that encapsulating the MMRII vaccine in an emulsion of the invention provided for improved recognition of the attenuated virus by a murine measles-specific antibody after heating at 37° C., as compared to the control formulation.

1-27. (canceled)

28. An emulsion comprising:
(i) an inner disperse aqueous phase comprising albumin, at least one benzoic acid derivative, and at least one salt;
(ii) a lipophilic phase comprising at least one polyunsaturated fatty acid, a nonionic surfactant, and a polyglycerol ester of a fatty acid and
(iii) optionally, an outer continuous aqueous phase comprising an aqueous solvent, a nonionic surfactant, and at least one salt,
wherein the lipophilic phase is an intermediate disperse lipophilic phase when the optional continuous aqueous phase is present, and wherein the lipophilic phase is an outer continuous lipophilic phase when the optional continuous aqueous phase is absent.

29. The emulsion of claim 28, wherein the emulsion is a W/O/W double emulsion comprising the inner disperse aqueous phase, the intermediate disperse lipophilic phase, and the outer continuous aqueous phase.

30. The emulsion of claim 28, wherein the at least one benzoic acid derivative is at least one compound independently selected from the group consisting of a compound of formula (I), a compound of formula (II), and a compound of formula (III): 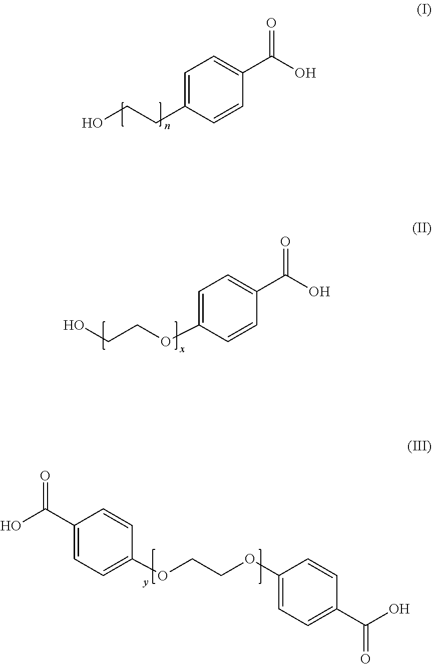
wherein each of n, x, and y is independently an integer of 1 to 12.

31. The emulsion of claim 28, wherein the at least one benzoic acid derivative is 1,8-bis(p-carboxyphenoxy)-3,6-dioxaoctane.

32. The emulsion of claim 28, wherein the emulsion is a W/O/W double emulsion comprising the inner disperse aqueous phase, the intermediate disperse lipophilic phase, and the outer continuous aqueous phase, and wherein the outer continuous aqueous phase further comprises a polyglycolide of formula (IV): 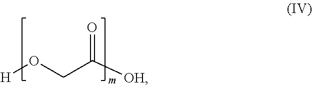
wherein m is an integer of 2 to 12.

33. The emulsion of claim 28, wherein the emulsion is a W/O/W double emulsion comprising the inner disperse aqueous phase, the intermediate disperse lipophilic phase, and the outer continuous aqueous phase, and wherein:
(i) the inner disperse aqueous phase comprises albumin, 1,8-bis(p-carboxyphenoxy)-3,6-dioxaoctane, and sodium chloride;
(ii) the intermediate disperse lipophilic phase comprises linoleic acid, sorbitan monooleate, and polyglycerol polyricinoleate; and
(iii) the outer continuous aqueous phase comprises aqueous ethanol, polysorbate 80, and sodium chloride.

34. The emulsion of claim 33, wherein a ratio by volume of the inner disperse aqueous phase:intermediate disperse lipophilic phase:outer continuous aqueous phase is 1:1:1 to 1:2:5.

35. The emulsion of claim 28, further comprising a substrate dispersed in the inner disperse aqueous phase.

36. The emulsion of claim 28, wherein the emulsion is a W/O/W double emulsion comprising the inner disperse aqueous phase, the intermediate disperse lipophilic phase, and the outer continuous aqueous phase further comprising a substrate dispersed in at least one of the inner disperse aqueous phase and intermediate disperse lipophilic phase.

37. The emulsion of claim 36, wherein the substrate is a heat-sensitive substrate.

38. The emulsion of claim 28, wherein at least 80% (w/v) of the emulsion is at least one of biodegradable and biocompatible.

39. A kit comprising:
(i) a first aqueous composition comprising albumin, at least one benzoic acid derivative, and at least one salt;
(ii) a lipophilic composition comprising at least one polyunsaturated fatty acid, a nonionic surfactant and a polyglycerol ester of a fatty acid; and
(iii) a second aqueous composition comprising an aqueous solvent, a nonionic surfactant, and at least one salt.

40. A method of improving thermal resistance of a substrate or thermally insulating a substrate by encapsulating the substrate in an emulsion, the method comprising:
(i) mixing the substrate with a first aqueous composition to form an aqueous mixture, the first aqueous composition comprising albumin, at least one benzoic acid derivative, and at least one salt;
(ii) mixing the aqueous mixture obtained in step (i) with a lipophilic composition to obtain a water-in-oil emulsion, the lipophilic composition comprising at least one polyunsaturated fatty acid, a nonionic surfactant and a polyglycerol ester of a fatty acid; and
(iii) optionally mixing the water-in-oil emulsion obtained in step (ii) with a second aqueous composition to obtain a water-oil-water emulsion, the second aqueous composition comprising an aqueous solvent, a nonionic surfactant, and at least one salt.

41. The method of claim 40, wherein the mixing is performed by a microfluidics device.

42. A method of thermally insulating a substrate or improving thermal resistance of a substrate, the method comprising:
(i) encapsulating the substrate in at least one phase of the emulsion of claim 28; or
(ii) surrounding the substrate with the emulsion of claim 28.

43. A layered composition comprising:
(i) a lipophilic layer comprising at least one polyunsaturated fatty acid, a nonionic surfactant and a polyglycerol ester of a fatty acid;
(ii) a first aqueous layer comprising an aqueous solvent, a nonionic surfactant, and at least one salt; and
(iii) optionally, a second aqueous layer comprising albumin, at least one benzoic acid derivative, and at least one salt, wherein when present, the second aqueous layer is separated from the first aqueous layer by the lipophilic layer.

44. A method of thermally insulating a substrate or improving thermal resistance of a substrate, the method comprising:
(i) encapsulating the substrate in at least one layer of the layered composition of claim 43; or
(ii) surrounding the substrate with the layered composition of claim 43.

45. A method of making the emulsion of claim 28, the method comprising:
(i) mixing a first aqueous composition with a lipophilic composition to obtain a water-in-oil emulsion, wherein the first aqueous composition comprises albumin, at least one benzoic acid derivative, and at least one salt, and the lipophilic composition comprises at least one polyunsaturated fatty acid, a nonionic surfactant and a polyglycerol ester of a fatty acid; and
(ii) optionally mixing the water-in-oil emulsion obtained in step (i) with a second aqueous composition to obtain a water-oil-water emulsion, the second aqueous composition comprising an aqueous solvent, a nonionic surfactant, and at least one salt.

46. The method of claim 45, wherein the mixing is performed by a microfluidics device.

47. The kit of claim 39 further comprising a microfluidic device for mixing (i), (ii) and (iii).
